(19) United States
(12) Patent Application Publication
(10) Pub. No.: US 2016/0174946 A1
(43) Pub. Date: Jun. 23, 2016

(54) SYSTEM FOR TRACKING FEMALE FERTILITY

(71) Applicant: Kindara, Inc., Boulder, CO (US)

(72) Inventors: William Sacks, Boulder, CO (US); Katherine Bicknell, Boulder, CO (US)

(73) Assignee: Kindara, Inc., Boulder, CO (US)

(21) Appl. No.: 15/059,150

(22) Filed: Mar. 2, 2016

Related U.S. Application Data
(63) Continuation of application No. 13/696,438, filed on Nov. 6, 2012, now abandoned.
(60) Provisional application No. 61/354,182, filed on Jun. 11, 2010.
(60) Provisional application No. 61/350,084, filed on Jun. 1, 2010.
(60) Provisional application No. 61/332,701, filed on May 7, 2010.

(51) Int. Cl. A61B 10/00; G06F 19/00

(52) U.S. Cl. CPC A61B 10/0012; G06F 19/3406; A61B 2010/0019; A61B 2010/0022

(57) ABSTRACT

Described herein are various principles related to collecting and charting fertility data for a female (e.g., female humans). A dedicated device may be used to collect fertility data regarding the female. The device may include a thermometer for collecting a body temperature of the female as well as an interface for collecting data regarding a consistency and/or amount of a cervical fluid of the female. Once collected by the dedicated device, the fertility data may be transmitted wirelessly from the dedicated device via a wireless network (e.g., a wireless local area network) to a server that collects fertility data. The fertility data may then be displayed in a graph that enables a user to make determinations about a fertility cycle of the female, including determinations about times of high fertility. For example, from viewing the relationship between two lines included in the graph, the user may make determinations regarding fertility.

### RELATED APPLICATIONS

This application claims priority under 35 U.S.C. §119(e) to U.S. Provisional Application Ser. No. 61/332,701, entitled “Wireless Fertility Tracking Thermometer,” filed on May 7, 2010, which is incorporated herein by reference in its entirety.

This application claims priority under 35 U.S.C. §119(e) to U.S. Provisional Application Ser. No. 61/350,084, entitled “Method To Display And Facilitate The Estimation Of Female Fertility For The Purposes Of Conception Or Contraception,” filed on Jun. 1, 2010, which is incorporated herein by reference in its entirety.

This application claims priority under 35 U.S.C. §119(e) to U.S. Provisional Application Ser. No. 61/354,182, entitled “System And Method To Measure And Track Female Fertility,” filed on Jun. 11, 2010, which is incorporated herein by reference in its entirety.

### BACKGROUND

1. Technical Field

Embodiments of the present invention relate to tracking fertility of females, particularly human females. Devices for collecting data on bodily conditions of a female are described as well as techniques for analyzing and displaying that data to determine fertility and/or a fertility cycle of the female.

2. Discussion of Related Art

Fertility management techniques are used for a variety of reasons. Some use fertility management techniques to aid in achieving a pregnancy, while others use the same techniques to avoid pregnancy. In some cases, fertility management techniques could be used in connection with in vitro fertilization or artificial insemination. For example, by identifying a fertility cycle of a woman or identifying the fertility cycles of two women (e.g., a donor and an acceptor), a time that a fertilized egg should be implanted can be identified. Additionally, fertility management techniques can be useful in identifying, managing, and (if applicable) treating menopause, pregnancy, and certain health conditions or disorders.

Many fertility management techniques rely on external factors, like hormone supplements, contraceptives, or information about average fertility cycles in a population of females (e.g., the average 28-day cycle for human females). Some techniques rely on information on characteristics of a particular female to determine fertility during the female's natural fertility cycle. One such fertility management technique is the Fertility Awareness Method (FAM). This technique relies on natural fluctuations of characteristics of the female body that correspond to fluctuations in fertility of the female. Levels of various hormones, the Basal Body Temperature (BBT) of the female, the position of the cervix, and consistency of the cervical fluid all may vary during the fertility cycle. For example, a female's cervical fluid consistency changes from a dry or sticky consistency during infertility, to a stretchy or wet consistency during a time of high fertility, while basal body temperature drops trends lower prior to ovulation and then rises and stays elevated following ovulation, in a given fertility cycle. By measuring and tracking these characteristics of the female body over the days (or, in some cases, months) of a cycle, a likely fertility at any one time can be determined and fertility over the span of the cycle can be tracked. From the tracking of fertility over time, the rhythm of the female's fertility cycle (which may deviate in minor or even major ways from an average for that species) can be determined.

### SUMMARY

In one aspect, there is provided a method for charting fertility data regarding a female. The method comprises receiving the fertility data regarding the female, the fertility data comprising a temperature of the female and at least a consistency of a cervical fluid of the female, and superimposing at least two two-dimensional coordinate systems in a single graph of a graphical user interface. The at least two two-dimensional coordinate systems comprise a first coordinate system having a temperature dimension and a time dimension and a second coordinate system having a cervical fluid dimension and a time dimension. The superimposing comprises identifying first coordinates corresponding to the temperature in the first coordinate system and identifying second coordinates corresponding to the cervical fluid consistency in the second coordinate system. Superimposing the at least two two-dimensional coordinate systems in the single graph comprises aligning the at least two two-dimensional coordinate systems such that coordinates indicating high fertility of the female in each of the at least two two-dimensional coordinate systems are closely aligned. The method further comprises displaying the fertility data in the graphical user interface.

In another aspect, there is provided an apparatus for accepting as input fertility data regarding a female. The apparatus comprises a housing, a thermometer, physically connected to the housing, to measure the basal body temperature of the female, and a user interface disposed in the housing to accept input from a user regarding a consistency and an amount of cervical fluid of the female. The user interface comprises at least one interaction element corresponding to a type of consistency of cervical fluid. The apparatus further comprises at least one storage medium to store fertility data received via the thermometer and/or the user interface in association with a time and/or date the fertility data is received.

In a further aspect, there is provided a system for managing fertility data regarding a female. The system comprises at least one device to accept as input fertility data regarding the female, each of the at least one device comprising a thermometer to measure the basal body temperature of the female, and a user interface to accept input regarding a consistency and an amount of cervical fluid of the female. The user interface comprises a plurality of interaction elements each corresponding to a type of consistency of cervical fluid. The system further comprises at least one server to store fertility data received from the at least one device and to generate a graph for a fertility cycle of the female. The graph comprises at least two lines corresponding to fertility data received from the at least one device and the at least two lines comprise a first line indicating variations in consistency and amount of cervical fluid of the female over time and a second line indicating variations in temperature of the female over time. In the system, the at least one device and the at least one server are connected via at least one communication network comprising a wireless network and the Internet.

In another aspect, there is provided a method of operating a fertility data collection device to collect fertility data for a female. The fertility data collection device comprises a thermometer and a user interface by which fertility data can be input. The method comprises measuring a temperature of the female with the thermometer of the fertility data collection device, receiving at least one input regarding a cervical fluid of the female via the user interface, the at least one input relating to a consistency and/or amount of the cervical fluid, and storing the temperature, time and/or date of the measuring of the temperature, and the at least one input in a storage medium of the fertility data collection device. The method further comprises wirelessly transmitting, from the fertility data collection device to a geographically remote computing device via a wireless network, the temperature and the at least one input.

In a further aspect, there is provided a method of distributing fertility data regarding a female. The method comprises receiving the fertility data regarding the female, the fertility data comprising at least one temperature of the female and at least one indication regarding a cervical fluid of the female, storing the fertility data in a fertility database in association with a user account associated with the female, restricting access to the fertility data on the fertility database to the user account, receiving an instruction from a user for the user account to share the fertility data with at least one second user account, and permitting access to the fertility data on the fertility database by the at least one second user account.

In another aspect, there is provided a method for charting fertility data regarding a female. The method comprises receiving the fertility data regarding the female, the fertility data a consistency of a cervical fluid of the female and a vaginal sensation of the female, and displaying the fertility data in a graphical user interface. The displaying comprises, in a graph of the graphical user interface, drawing a line in a two-dimensional coordinate system having a cervical fluid dimension and a time dimension, the drawing comprising identifying coordinates corresponding to the cervical fluid consistency in the two-dimensional coordinate system. The displaying also comprises, when the vaginal sensation indicates that the female is fertile at a particular time, shading at least a portion of the graph of the graphical user interface corresponding to the particular time, the portion of the graph being between the line and a horizontal axis of the graph.

The foregoing is a non-limiting summary of the invention, which is defined by the attached claims.

### BRIEF DESCRIPTION OF DRAWINGS

The accompanying drawing figures are not intended to be drawn to scale. Each identical or nearly identical component that is illustrated in various figures is represented by a like numeral. For purposes of clarity, nom every component may be labeled in every drawing figure. In the drawings:

FIG. 1 is an example of a graph of fertility data that may be produced in some embodiments;

FIG. 2 is another example of a graph of fertility data showing fertility data for a female across three fertility cycles for the female:

FIG. 3 is a sketch of an exemplary user interface that may be implemented in some embodiments to collect and/or display fertility data for a female;

FIGS. 4A and 4B are, respectively, front and back plan views of one example of a fertility data collection device;

FIGS. 5A and 5B are, respectively, top and side partially cutaway plan views illustrating internal components of one example of a fertility data collection device;

FIGS. 6A and 6B are, respectively, front and back plan views of a second example of a fertility data collection device;

FIG. 7 is a flowchart of one exemplary method for initializing a fertility data collection device;

FIG. 8 is a screenshot of one exemplary user interface that may be used to configure a fertility data collection device;

FIG. 9 is another screenshot of the exemplary user interface of FIG. 8 that may be used to configure a fertility data collection device;

FIG. 10 is a flowchart of one exemplary method of operating a fertility data collection device to collect fertility data and transmit the fertility data to a data destination;

FIG. 11 is a flowchart of one exemplary method of creating a graph from fertility data received at a data destination;

FIG. 12 is a schematic illustration of one exemplary system for collecting and tracking fertility data; and

FIG. 13 is a schematic illustration of a second exemplary system for collecting and tracking fertility data.

### DETAILED DESCRIPTION

Many fertility awareness techniques as well as tools for collecting data exist. For example, paper templates exist for people to chart collected data regarding fertility characteristics. As another example, some electronic apparatuses exist for collecting and tracking information about some fertility characteristics.

Existing tools, however, are awkward and inconvenient for users. This inconvenience deters consistent use of these tools, which undermines the effectiveness of the tools. For example, to collect accurate measurements of BBT, the temperature should be read immediately upon waking from a long stretch of sleep and at the same time each day. To place these measurements into a chart, the user is required to manually enter the data into a computing device, connect a sensor to a computing device with a wire, or manually sketch the data on a paper chart. Requiring such extra manual steps is undesirable, at least because this inconveniences the users who take the measurements at night or in the morning just after waking from sleep.

Applicants have recognized and appreciated that there would be advantages to systems that facilitate the collection of fertility data, including information on bodily characteristics of a female. Advantages also could be offered through improvements in charting fertility data, allowing users to better understand the fertility data that have been collected and to make their own determinations regarding fertility cycles.

In view of the foregoing, described herein are various embodiments related to collecting and charting fertility data for a female. These embodiments may be used for collecting and charting fertility data of human females as well as females of other species. In some embodiments, a user (who may be the female or a different person) collect fertility data regarding the female using one or more dedicated devices. The dedicated device may include a thermometer for collecting the BBT of the female as well as an interface for collecting data regarding other characteristics of the female. The other characteristics may include, for example, consistency of a cervical fluid of the female, an amount of the cervical fluid, and a sensation of the female's vagina (e.g., a wet or dry sensation, or whether there is a wet sensation). In some embodiments, the fertility data, once collected by the dedicated device, may be transmitted from the dedicated device via a wireless local area network (WLAN) and via the Internet to a server that collects fertility data. The fertility data may be associated with a user account and with any previously-collected fertility data for that female. Additionally, in some embodiments, the fertility data for that female (both new and previously-collected) may be charted such that a graph may be provided to a user via a graphical user interface. The graphical user interface enables the user to make determinations about the fertility cycle of the female, including determinations about times of high fertility and menstruation. For example, from viewing the relationship between two lines included in the graph, the user may make determinations regarding fertility.

Some of the fertility characteristics that are tracked in embodiments are known in the art. For example, the Basal Body Temperature (BBT) of a female is the steady, resting temperature of the female, such as the temperature upon waking from sleep. As will be appreciated from further description below, the BBT of the female is typically low in a period of time (e.g., several days or weeks) leading up to ovulation and rises immediately following ovulation. The BBT typically remains high throughout the remainder of the cycle, until a female menstruates. Cervical fluid consistency refers to a viscosity of the cervical fluid produced by the female, which may be subjectively measured by a user on a scale including categories for “sticky.” “creamy,” and “egg-white.” The cervical fluid can be rested by a user in various ways, including through testing a stickiness of the cervical fluid between the user's fingers. Similarly, cervical fluid amount refers to a volume of the cervical fluid for the female, which may be subjectively measured by the user on a scale of small, medium, and large amounts. As the amounts generated by females may vary between females, the amounts may be measured by the user relative to the amounts typically generated by the female being measured. As will be appreciated from further description below, as a female becomes more fertile, the viscosity and amount of cervical fluid will increase and, following ovulation, the viscosity and amount of cervical fluid will decrease. Vaginal sensation is another fertility characteristic that may be tracked. In some cases, the vaginal sensation may be measured as “wet” and “dry” or as “wet” and not “wet.” Vaginal sensation refers to the female's subjective impression of the cervical fluid present in the vagina at the time of measurement and whether the cervical fluid is causing the female's vagina to feel “wet.” This is the female's impression of the vagina during her normal routine, without touching the vagina, and under normal circumstances rather than during or following a period of arousal. A “wet” vaginal sensation refers to the female's feeling of lubrication (or extra lubrication) along the internal labia (the labia minora) of the vagina, while a “dry” or not “wet” vaginal sensation refers to a lack of such a feeling of lubrication. Some females may compare this sensation to a feeling of incontinence or a feeling that menstruation is about to begin. In a time period immediately prior to ovulation (e.g., several days prior to ovulation) and during ovulation, the vaginal sensation for the female may become “wet,” and following ovulation and until menstruation begins the vaginal sensation for the female may be “dry” or not “wet.” While in some embodiments, the vaginal sensation may be tracked using the binary “wet”/“dry” measurement scale, in other embodiments vaginal sensation may be measured using three, four, or more categories. For example, vaginal sensation could be measured as “dry” indicating no sensation of lubrication, “wet” indicating some sensation, and “lubricated” indicating a lubricated and/or slippery sensation.

Embodiments may track some, all, or none of these exemplary fertility characteristics. Embodiments are not limited to tracking any particular fertility characteristics. Described below are various non-limiting, illustrative embodiments for tracking female fertility.

In some embodiments, a graph may be used to chart fertility data including temperatures, consistencies of cervical fluid, amounts of cervical fluid, and vaginal sensation over time. The graph includes two two-dimensional coordinate systems that are superimposed: a first coordinate system for temperature and time and a second coordinate system for cervical fluid and time. The coordinate systems are superimposed such that lines drawn in each coordinate system can be displayed together and a relationship between them can be easily identified by a user.

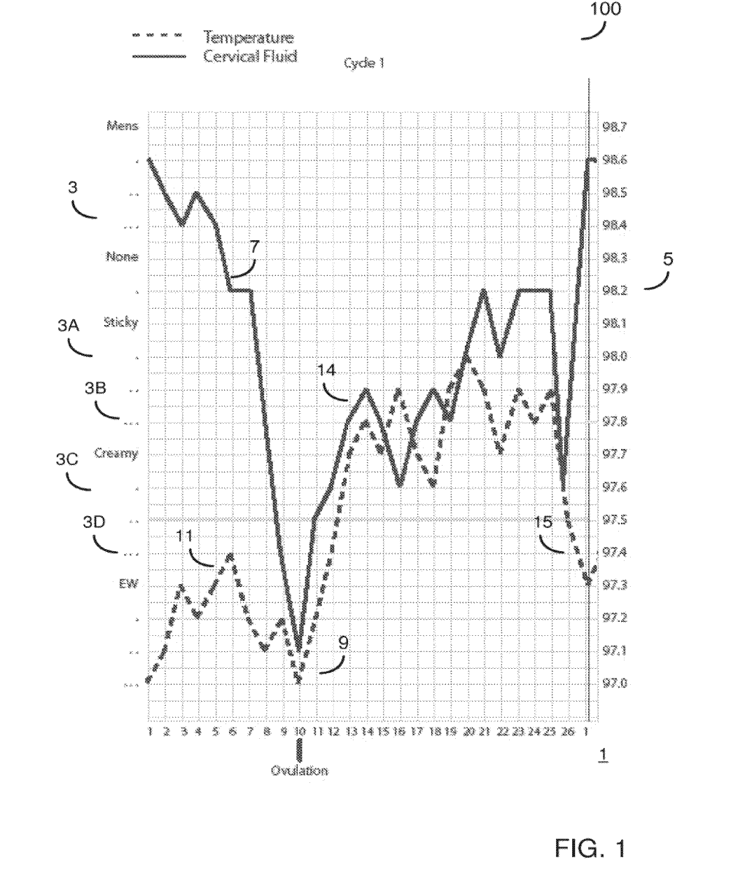

FIG. 1 illustrates one exemplary embodiment of a graph 100 that may be presented to a user to display fertility data for a female. The fertility data that may be displayed in the graph 100 includes data relating to the BBT for the female, consistency of the cervical fluid of the female, and the amount of the cervical fluid of the female.

Graph 100 includes fertility data as a function of time which is collected for a user over the course of one fertility cycle. The horizontal axis or abscissa of graph 24 is time, shown along the bottom 1 of the graph 100 as the days of a fertility cycle (a 26-day cycle in the exemplary cycle illustrated in FIG. 1). The graph 100 also includes two different ordinates or vertical axes 3 and 5, each of which relates to a different type of fertility data that may be displayed in graph 100.

The graph 100 can be used to display data relating to two or more coordinate systems. Each two-dimensional coordinate system in the graph relates to a fertility characteristic in one dimension and time in a second dimension, so that fertility characteristics can be graphed over time. In one coordinate system, temperature is plotted versus time. In another coordinate system, information about cervical fluid is plotted versus time. These coordinate systems can be organized in any suitable manner.

In some embodiments, the coordinate system for cervical fluid may be arranged so as to accent increasing fertility. For example, the sections of the coordinate system corresponding to each consistency of cervical fluid may have arranged within them subsections corresponding to amounts of that consistency of cervical fluid. The coordinate system may be configured so that a distance between two subsections of the same consistency (e.g., two amounts of a same consistency) is smaller than the distance between two sections of different consistencies. For example, the distance between a medium amount and a large amount of a “Sticky” consistency type may be smaller than the distance between a large amount of “Sticky” and a small amount of “Creamy.” The coordinate system may be arranged in this way to accent the increase of fertility that corresponds to the increase in viscosity of the cervical fluid. The difference between types of cervical fluid is a better indicator of fertility than merely a change in amounts of the same type. The difference between sections along the cervical fluid axis may be implemented in any suitable manner. In some graphs, for example, a single unit of the cervical fluid axis may separate amounts of the same consistency type and two units of the cervical fluid axis may separate the sections corresponding to different consistency types.

Accordingly, the vertical axis 3 relates to observations of the cervical fluid of the female to whom the fertility data belongs. The coordinates of the vertical axis 3 are arranged according to their relationship to fertility of the female. Coordinate positions near the top of the axis 3 correlate to low fertility while coordinate positions near the bottom of the axis 3 correlate to high fertility. The axis 3 displays both consistencies (or types) and amounts of cervical fluid. To display both the consistencies and the amounts, the axis 3 is divided into multiple sections, each based on consistency. Each of these sections is associated with a commonly-understood label for consistencies. In the exemplary embodiment of FIG. 1, there are five sections labeled menstruation, none, sticky, creamy, and egg-white. Any other suitable labels may be used depending on what is customary for a particular user or for a particular country. Each of the sections is subdivided into subsections related to the amount of the cervical fluid. In the embodiment of FIG. 1, the amounts are displayed at three levels-small, medium, and large—but any suitable labels may be used to indicate the same information. The small amount typically may be the top line in each section, while the large amount may be the bottommost line in each section. For example, “Sticky” section 3A in the graph includes three subsections 3B relating to different amounts of a “Sticky” cervical fluid, and “Creamy” section 3C includes three subsections 3D for different amounts of “Creamy” cervical fluid.

The vertical axis 5 relates to the BBT of the female. The temperature is illustrated in the graph of FIG. 1 in degrees Fahrenheit and on a scale of 97.0-98.7 degrees, but any suitable temperature system and scale may be used. In some embodiments, the scale of axis 5 may be modified to reflect the temperatures previously observed in that person's fertility data, so that the range of temperatures displayed in the graph 100 will correspond to the range of temperatures that have been observed historically.

When fertility data are received, positions corresponding to the fertility data in each of the two coordinate systems are located. For temperature, a horizontal point in the temperature coordinate system corresponding to the time the temperature was collected and a vertical value of the temperature are located. For cervical fluid, a vertical section corresponding to the consistency is identified, and within that section a subsection corresponding to the amount is identified. A horizontal coordinate corresponding to a time the cervical fluid was measured is identified, so that a point in the cervical fluid coordinate system is identified. A line 7 connecting the cervical fluid observation points and a line 11 connecting the temperature reading points can be drawn.

The lines 7 and 11 of the graph 100 can be observed by a user to determine a fertility level of the female. Large amounts of cervical fluid and cervical fluid that is more viscous are indicative of high fertility. Thus, in the graph 100 as the line 7 extends downward toward the bottom of the graph 100 (indicated by reference number 9), increasing fertility is indicated. Additionally, a lower BBT is indicative of a higher fertility. While the line 11 remains near the bottom of the graph 100, higher fertility is indicated.

Each of the lines 7, 11 may individually provide some indication of fertility of a female, while lines 7, 11 together provide a more precise indication of fertility. Before ovulation, the closer together lines 7, 11 are, the more likely the female is to be fertile. More particularly, when the cervical fluid line 7 is higher as shown in FIG. 1 and the temperature line 11 is low as shown in FIG. 1, the female is likely to be in a pre-ovulatory infertile phase. When both lines 7, 11 approach the bottom of the graph, as shown in FIG. 1, the female is more likely to be in a fertile phase of the cycle. Ovulation is indicated when the lines 7, 11 rise steeply together toward the top of graph 100, as indicated by the rise 14 in FIG. 1. When both lines 7, 11 are high, the female is likely in a post-ovulatory infertile phase. Temperature line 11 drops again at the start of menstruation (indicated by reference number 15).

The superimposition of the two coordinate systems in the graph 100, as well as the use of two lines corresponding to temperature and cervical fluid, allows a user to quickly and easily learn about the fertility of the female to whom the fertility data relates. Both systems demonstrate higher fertility near the bottom of the graph 100, and lower fertility towards the top of graph 100. Of course, those positions could be reversed so that greater fertility of both coordinate systems occurs toward the top of the graph and lower fertility is indicated as the curve extends toward the bottom of the graph.

Embodiments that graph the fertility data in the manner of FIG. 1 therefore offer advantages over conventional systems that graph only the temperature and provide information about cervical fluid separately, such as by merely labeling days with cervical fluid amounts and consistencies, or systems that graph cervical fluid as a bar graph.

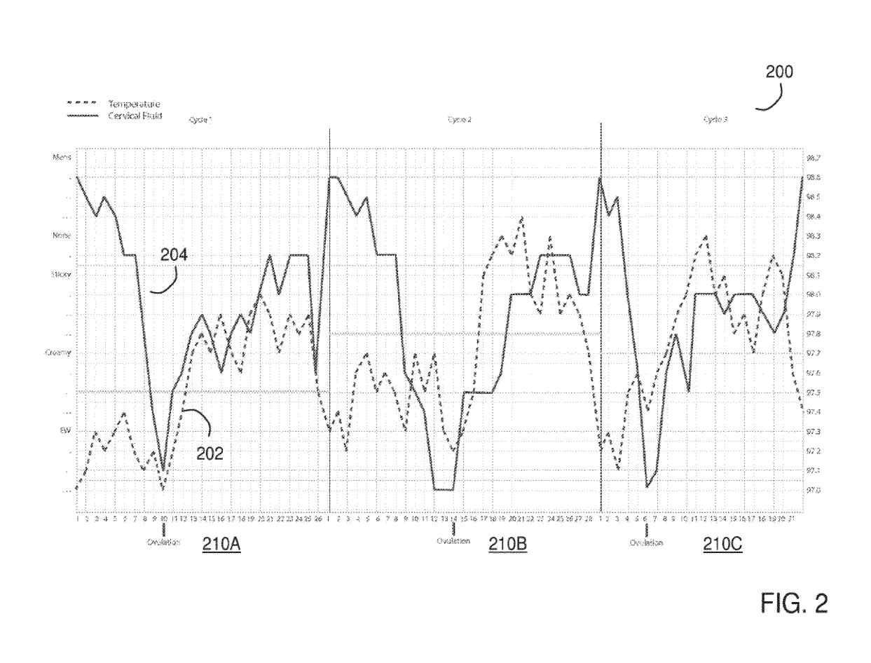

FIG. 2 shows another graph 200 that includes fertility information related to BBT and cervical fluid amounts and consistencies. In particular, graph 200 includes fertility information charted over three consecutive cycles 210A, 210B, 210C for a female to illustrate the relationship between temperature line 202 and cervical fluid line 204 over time. As discussed in detail above, the line 202 and line 204 both have low values in their respective coordinate systems at a time corresponding to high fertility of the female to whom the fertility characteristics relate. Accordingly, ovulation of the female is marked at three locations in the graph 200 that correspond to times that the lines 202, 204 both have low values.

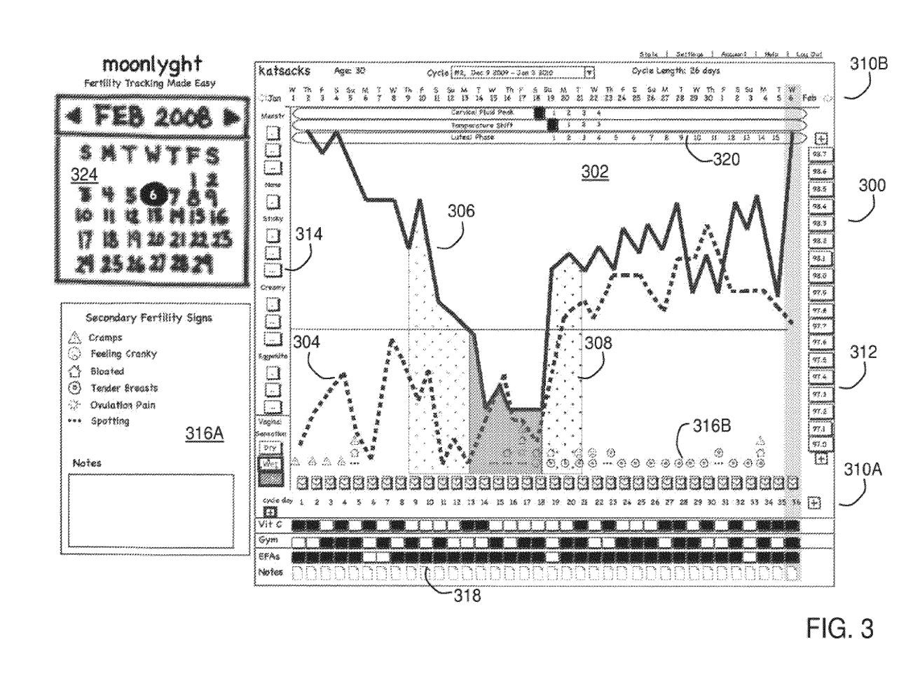

FIG. 3 includes a sketch 300 of one exemplary user interface that may be used in some embodiments to convey fertility information to a user. In particular, sketch 300 shows an example of a web interface that may be provided by the Moonlyght service provided by Conscious Cycles, LLC, of New York, N.Y. The Moonlyght service may collect fertility information for a female in any suitable manner, including using some exemplary input devices described below or through the web interface illustrated in sketch 300. The Moonlyght service presents the fertility information to a user in a way that enables the user to track the female's fertility over time and determine when the female is or is not fertile.

The sketch 300 shows the interface including a graph 302, which is similar in some ways to the graph 100 of FIG. 1. The graph 302 includes a line 304 indicating a BBT of a female over time and a line 306 indicating a consistency and amount of cervical fluid of the female over time. In addition, graph 302 indicates a vaginal sensation of the female over time. In particular, in graph 302 a region under the line 306 can be shaded, as in the regions 308, to indicate a vaginal sensation over time. In the example of FIG. 3, a lack of shading under the line 306 indicates a “dry” sensation, a dotted shading indicates a “wet” sensation, and a solid shading indicates a “lubrication” sensation.

As above in the example of graph 100 of FIG. 1, the lines 304, 306 and shading 308 of the graph 302 are illustrated along a time axis, shown in the sketch 300 as two axes 310A, 310B. Axis 310A tracks time as a function of days of a fertility cycle of the female, from the start of menstruation on day 1 through fertility until the start of the menstruation of the next fertility cycle. Axis 310B tracks time as a date, with days of the month illustrated along the axis. Time is also a function of each of the two-dimensional coordinate systems in which the lines 304, 306 appear. Temperature line 304 appears in a two-dimensional coordinate system including temperature (as shown by axis 312) and time and cervical fluid line 306 appears in a two-dimensional coordinate system including cervical fluid amounts and consistencies (as shown by axis 314) and time. As above in the graph 100 of FIG. 1, the axis 314 is divided into five sections relating to cervical fluid: menstruation, none, sticky, creamy, and egg-white. Each of the sections is also divided into subsections relating to amounts of each consistency.

The interface illustrated in sketch 300 also includes areas that track other information for users, including secondary fertility characteristics for the female. Secondary fertility characteristics include those characteristics that may be related in some way to fertility, but may not be, by themselves, reliable factors by which to track fertility. For example, some secondary characteristics may not be consistent between females or may not be consistent between cycles of a single female. As illustrated in the sketch 300, these secondary fertility characteristics include cramping, feeling cranky, feeling bloated, feeling tenderness in breasts, feeling pain associated with ovulation, and spotting. The secondary fertility characteristic may be selected for a particular day in area 316A of the interface of sketch 300 and may be displayed associated with the selected day in area 316B of the interface. Each characteristic may be associated with an icon that, when the characteristic is selected by the user for a given day to indicate that the characteristic was experienced by the female that day, is displayed in the area 316B.

Sketch 300 also shows an area 318 by which a user can track any other factors of interest to the user. These factors may be tracked by the user to aid the user in determining whether the fertility of the female is impacted in any way by the factors. In the example of sketch 300, a user has indicated which days a vitamin C supplement was administered to the female and which days the female exercised. This is shown by a shading of boxes associated with days that the supplement was taken or an exercise was performed and a lack of shading on other days. In addition, boxes with heart icons are used in the area 318 to indicate whether the female engaged in sexual intercourse on any particular day, such that the heart may be unshaded on days without intercourse and shaded on days where there was intercourse. Any suitable factors may be tracked in any suitable manner, as embodiments are not limited in this respect. Accordingly, while the examples shown in FIG. 3 are binary factors with only “yes” or “no” states, other factors may be tracked with any suitable number of states and may be displayed in an interface in any suitable fashion.

The interface of the sketch 300 also includes functionality for a user to track or make predictions about the fertility of the female to which the fertility data relates, as shown in area 320. The area 320 includes three different prediction timelines that can be created based on user input. The first timeline is based off user input regarding which day is the peak for cervical fluid amount and consistency—which corresponds to the lowest point in the cycle for line 306. For four days following the peak cervical fluid consistency/amount, the female is likely to be fertile, which is indicated by the four days following the shaded box indicating which day is the peak cervical fluid consistency/amount. After the evening of the fourth day following the peak day, the female is considered infertile. The second timeline is similar and is based on temperature. For three days following the spike in temperature that follows ovulation, the female is likely to be fertile. After the evening of the third day following a sustained temperature shift, the female is considered infertile. Accordingly, the second timeline indicates three days following a shaded box that indicates which day includes the rise in the female's temperature. The last timeline shows the time following ovulation and until the next period of menstruation. This is the luteal phase, which corresponds to the expected length of time until the next menstruation. For an average human female, this will be between 12 and 16 days. Though, this time period may vary between human females or between cycles of a human female. The luteal phase can be used by the user to track when the next menstruation for the female may be. The luteal phase can also be used to determine some possible conditions of the human, such as a pregnancy if the luteal phase extends beyond the average time period or hormone deficiencies if the luteal phase is shorter than expected.

Graph 100 of FIG. 1, graph 200 of FIG. 2, graph 302 of FIG. 3, or similar graphs may be produced in any suitable manner based on fertility data received from any suitable source. As discussed in greater detail below, a server may receive fertility data via the Internet, produce the graph 100 from the fertility data, and provide the graph 100 as part of a web page. The fertility data may be received by the server from any suitable source.

In some embodiments, a user interface like the interface illustrated in sketch 300 of FIG. 3 may be used to input fertility data. For example, a user may select a day in the graph 302 or via the calendar 324 and then use other buttons in the interface to input data. The indications of amounts of cervical fluid consistencies can be implemented as buttons to input a particular amount of cervical fluid of a particular consistency for the day and the temperatures can be similarly implemented as buttons. In addition, the icons for intercourse, secondary fertility signs, and other fertility factors may be similarly implemented as buttons. In this way, fertility data can be received as input and used to produce the graph 302 illustrated in FIG. 3.

In some embodiments, an application for a mobile computing environment, such as an application for the Apple iOS platform or the Google Android platform, may be used to collect fertility data. For example, a user may input to a software application via one or more interfaces various fertility characteristics as fertility data to be transmitted to a server and used to produce the graph 302 illustrated in FIG. 3. In some embodiments, the user may input the fertility characteristics as alphanumeric text to the software application. In other embodiments, the user may additionally or alternatively input the fertility characteristics using sensing hardware or other hardware designed to interact with the software application and/or with the iOS or Android platform. For example, the user may input fertility characteristics using a thermometer that interacts with the mobile computing environment.

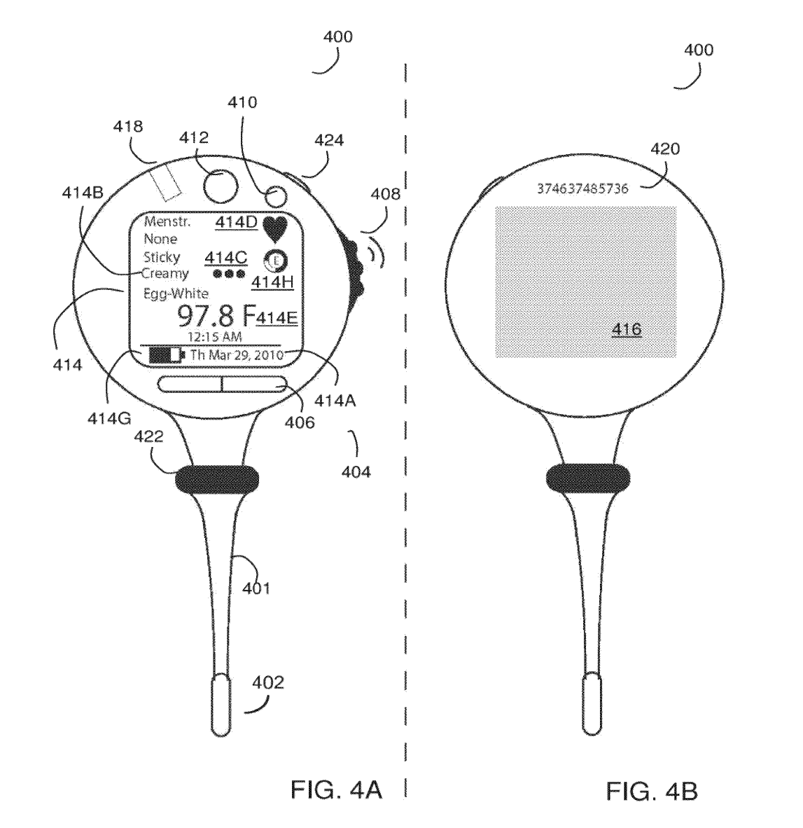

In some other embodiments, the server may additionally or alternatively receive the fertility data from one or more dedicated devices that collect fertility data, FIGS. 4A and 4B illustrate one example of a device 400 for collecting and sending fertility data. The fertility data collection device 400 includes a housing 401, which could be formed of plastic or any other suitable material, and in which are disposed various components for collecting and transmitting fertility data. Device 400 includes a thermometer 402 for measuring the BBT of the female as well as a user interface 404. The user interface 404 may include interaction elements 406, 408, 410, 412, 424 as well as a display 414. The interaction elements provide various inputs to the device 400, each of which is discussed below. The device 400 may also be branded with a serial number 420, which may be stored in memory and may be transmitted from the device 400 along with fertility data.

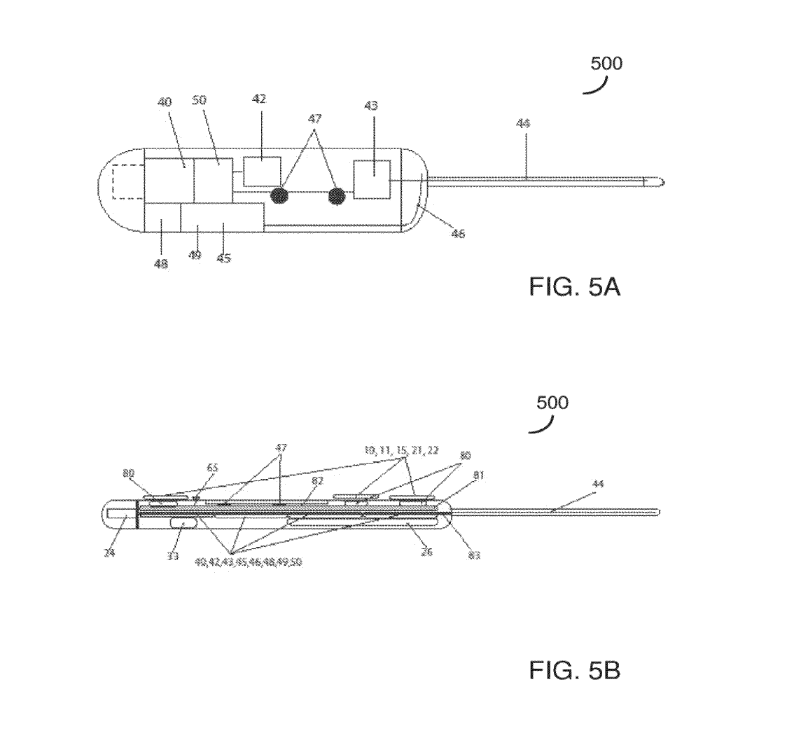

The device may include at least one storage medium (not illustrated), such as a memory, in which fertility data received from the thermometer 402 or the interface 404 may be stored. The device 400 may further include a battery (not illustrated) interior to the housing and a solar cell 416 for charging the battery using solar energy. The device 400 also may include two components for transmitting fertility data: a wired communication port 418 and a wireless transmitter interior to the housing (discussed below in connection with FIGS. 5A and 5B). The wired communication port 418 may be any suitable port using any suitable wired protocol, including Universal Serial Bus (USB). For example, the port 418 may be a mini-USB port. Similarly, the wireless transmitter may be any suitable transmitter for communicating using any suitable ad hoc or infrastructure wireless network. For example, the wireless transmitter may be adapted to communicate via a wireless local area network (WLAN), like a home wireless network, and/or via a wireless wide area network (WWAN), like a cellular communication network. The device 400 may be adapted to communicate any fertility data collected by the device 400 from the thermometer 402 or the interface 404 via either of the port 418 or the wireless transmitter.

The user interface 404 may include the interaction elements 406, 408, 410, 412, 424 each of which can be used to control the device 400 and/or to input data to the device 400. The interaction elements may be implemented in any suitable manner, including as buttons and/or scroll wheels. In some embodiments that use buttons, each of the buttons may include a unique raised dimple shape at its apex to distinguish one button from another in dim light.

Button 424 may be operated as an on/off button to allow a user to manually turn the device 400 on and off.

Buttons 406 may be used to select a time and/or date to associate with input data or to display previously-input data. When fertility data is input to the device 400 and stored and/or transmitted by the device 400, the fertility data is associated with a time and date, so that the fertility data can be displayed in a graph with a time coordinate (such as graph 100 of FIG. 1). Through operation of buttons 406, data also can be input for a time in the past, such as by inputting data for an observation of cervical fluid consistency and amount made on a previous day. As the buttons 406 are operated, a portion 414A of display 414 may be updated to show the currently-selected time and/or date.

Wheel button 408 may be used to input information regarding cervical fluid observations. In the embodiment of FIGS. 4A and 4B, information regarding five different types of cervical fluid can be input, each of which corresponds to the five types discussed above in connection with FIG. 1: menstruation, none, sticky, creamy, and egg-white. As mentioned above, any suitable labels for consistencies may be used, and more or fewer categories of consistency may be used. Operating the wheel button 408 by scrolling the wheel up or down allows the user to scroll through the list displayed in area 414B of the display 414 and, by selecting, to input a consistency of cervical fluid for the female. The cervical fluid may be observed by the user, separate from the device 400, and the user selects a consistency that the user believes most closely corresponds to the observed cervical fluid (e.g., is the cervical fluid more “sticky” or is it more like “egg-white”). The wheel button 408 may also be used to input amounts of the cervical fluid, by pushing the wheel button inward, toward the center of the device 400. Repeated operation of the button cycles through amounts of the selected consistency of cervical fluid. In the embodiment of FIGS. 4A and 4B, the eligible amounts are small, medium, and large amounts, although any suitable labels or numbers of amounts may be used. As the wheel button 408 is operated, the display 414 is updated. As shown in FIG. 4A, in response to pushing the wheel button 408 three times while the “Creamy” consistency is selected, a portion 414C of the display 414 shows three dots, which corresponds to a large amount of a “Creamy”-type cervical fluid. In the example of FIGS. 4A and 4B, two dots would indicate a medium amount and one dot would indicate a small amount.

Button 410 may be used to input information regarding sexual intercourse engaged in by the female, as well as whether the intercourse was protected or unprotected (e.g., through use of condoms). Repeated operation of the button 410 would cycle through protected, unprotected, and no intercourse. As the button is operated, a portion 414D of the display 414 is correspondingly updated with icons corresponding to those inputs. A solid heart in portion 414D indicates unprotected intercourse, an outline of a heart in portion 414D indicates protected intercourse, and no icon in portion 414D indicates no intercourse.

The display 414 may also include a portion 414E that displays a temperature for the female measured by the thermometer 402, as well as a portion 414G that indicates a current charge of a battery of the device 400.

Fertility data—including temperature, consistencies and amounts of cervical fluid, and intercourse data, all in association with a date and time—may be stored in a memory of the device 400 until the fertility data is transmitted from the device 400 via wired port 418 or the wireless transmitter via communication networks and/or the Internet to a remote server, such as a server that will produce a graph similar to graph 100 of FIG. 1.

When the device 400 is in an on state, operation of the button 412 may cause the device to transmit any fertility data stored by the device 400 wired and/or wirelessly. In some embodiments, the transmission may be done wirelessly by default and via a wired connection with a connection is made via the port 418. When the button 412 is operated to transmit the fertility data, a portion 414H of the display 414 may update with a circular progress bar showing progress of the transmission. If there is an error during the transmission, the portion 414H will display an error indicator, shown in FIG. 4A as an “E.” In some embodiments, the display 414 may provide more information to a user about the error, such as in response to a request from a user, while in other embodiments a user may connect the device 400 to a computing device via the port 418 to receive more information about the error.

In some embodiments, the thermometer 402 is designed to be used as an oral thermometer. The thermometer end of the device 400 therefore would be held in the mouth of the female while measuring the temperature. The device 400 may be arranged to be held easily in the mouth. For example, the device 400 may include a structure 422 that can be gripped by the teeth and/or lips while the temperature is being measured. Further, a center of gravity of the device 400 may be positioned proximate to the structure 422, to reduce torque on the thermometer 402 and make the device 400 easier to be held by the teeth. The center of gravity may be placed near the structure 422 in any suitable manner, such as by arranging heavier internal components, like the battery, closely adjacent to the structure 422. The center of gravity of the device 400 may be any distance from the structure 422 that enables the device 400 to be held in the mouth comfortably while taking a temperature, without the device 400 creating an uncomfortable torque on the mouth.

The fertility data collection device may include various internal structures and components, such as those shown in FIGS. 5A and 5. The components illustrated in FIGS. 5A and 5B may be used with the embodiment of FIGS. 4A and 4B, or with different embodiments.

FIG. 5A is a schematic diagram of the internal electronic components of the device 500. The device 500 may contain a circuit board 81 to which is connected at least one programmable processor 50, programmed with instructions for operating the device 500, a storage medium 40 (e.g., a flash memory), a display driver circuit 42 to control a display screen (not illustrated) to display data to a user, and a thermometer circuit 43. The thermometer circuit 43 is connected to a temperature sensor 44 and interprets signals transmitted by the sensor 44 indicative of a measured temperature. In response to operation of the device by a user, such as via user interface 404 of the device 400 of FIGS. 4A and 4B, the processor 50 may cause the thermometer circuit 43 to collect thermometer data, the storage medium 44 to store the data, and the display driver circuit 42 to display information on the display screen.

The main circuit board 81, programmable processor(s) 50, and storage medium also may be connected to communication components of the device 500. The communication components may include, for example, a USB driver circuit 48 to transmit data via a wired connection and a wireless transmitter/receiver 45 (e.g., a transceiver) connected to an antenna 46. In some embodiments, the wireless transmitter/receiver 45 may be adapted to communicate via one of a wireless local area network (WLAN) (e.g., via one of the Institute of Electrical and Electronics (IEEE) 802.11 family of protocols), a wireless personal area network (WPAN) (e.g., via Bluetooth), or a wireless wide area network (WWAN) (e.g., a cellular network). In alternative embodiments, the device 500 may include more than one transmitter/receiver or a transmitter/receiver 45 able to communicate via multiple protocols (e.g., both a WLAN and Bluetooth transmitter/receiver). In some embodiments in which the device 500 communicates via a WWAN, the device 500 may also contain a Subscriber Identity Module (SIM) card 49 that contains identifying information for the device 500 that can be transmitted to the network, as is known to those of skill in the art.

The programmable processor 50 also may be connected to and control components of a user interface, including a speaker for audible user feedback and two lights 47 for backlighting a display screen.

In some embodiments, some or all the aforementioned components may be included on a single custom designed circuit board 81.

FIG. 5B shows a cutaway side view of the device 500 of FIG. 5A. External button covers 10, 11, 15, 21, and 22 of the device 300 each may be associated with and/or connected to buttons 80, which are mounted to the circuit board 81. The aforementioned internal components 40, 42, 43, 45, 46, 48, 49, and 50 of FIG. SA also may be connected to the circuit board 81.

Additionally, a battery 26 and speaker 33 may be connected to the circuit board 81. USB port 24 also may be connected to the circuit board 81. Screen backlights 47 and screen 82 also may be connected to the main board 81.

The internal components illustrated in FIG. 5B may be shaped and arranged to fit securely inside two parts of a main body of the device 500. The two parts may be made of molded plastic or of any other suitable material, and may clip together along a seam 83 to permanently or semi-permanently close and hold all the internal components in place.

It should be appreciated that fertility data collection devices can be implemented in any suitable manner to collect any suitable type or types of fertility data for a female. Embodiments are not limited to operating with or collecting any particular type(s) of fertility data and are not limited to operating with fertility data collection devices that collect any particular type(s) of fertility data. In some embodiments, fertility data may include BBT, cervical fluid consistency, cervical fluid amount, and vaginal sensation and a fertility data collection device or other input mechanism may collect fertility data regarding each of these fertility characteristics. In other embodiments, however, one or more of these fertility characteristics may not be collected at all or may not be collected by a fertility data collection device.

For example, the exemplary device 400 of FIGS. 4A and 4B was configured to collect fertility data regarding BBT, cervical fluid consistency, and cervical fluid amounts, but was not configured to collect fertility data regarding vaginal sensation. In some embodiments, the device 400 may be configured to additionally or alternatively collect fertility data regarding vaginal sensation and/or fertility data regarding other characteristics.

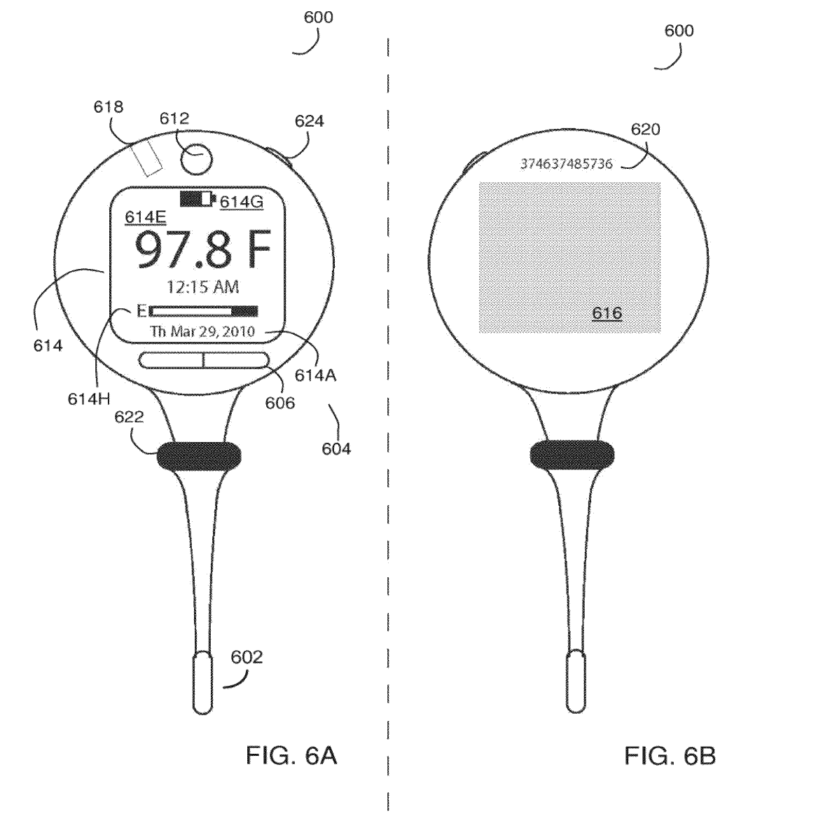

In other embodiments, a system for tracking a female's fertility may receive less fertility data from a dedicated fertility data collection device. FIGS. 6A and 6B show an example of such a fertility data collection device.

FIGS. 6A and 6B show plan views of a fertility data collection device 600. The device 600 is similar in some ways to the device 400 of FIGS. 4A and 4B. For example, the device 600 includes a thermometer 602, time selection buttons 606, transmission button 612, display 614, solar panel 616, port 618, serial number 620, and on/off button 624 that may be implemented similarly to corresponding elements of the device 400 discussed above. Device 600 differs from the device 400, though, in that the user interface 604 is configured to accept fewer types of fertility data as input than the interface 404 of device 400. More particularly, the interface 604 can be used to select a date/time, displayed in area 614A, and can be used to display, in area 614E, a temperature of the female collected using the thermometer 602. The display 614 also includes an area 614G identifying a battery power and an area 614H that displays a transmission status when the transmission button 612 is operated. The interface 604 of device 600 does not, however, include interface elements to permit the input fertility data relating to cervical fluid amounts or consistencies. Accordingly, the device 600 can be used by a user only to collect fertility data regarding a BBT of the female and a date and time the BBT was collected. In embodiments that operate with the device 600, additional fertility data regarding the female (e.g., cervical fluid amounts and consistencies, vaginal sensation, secondary fertility characteristics, etc.) may be input in other ways, such as via a web or mobile interface.

In some embodiments that operate with a fertility data collection device, the fertility data collection device may be used to collect fertility data from a female without requiring initialization of the fertility data collection device or of a separate computing device to be used with the fertility data collection device. In other embodiments, however, an initialization process may be carried out for one or both of a fertility data collection device and a separate computing device.

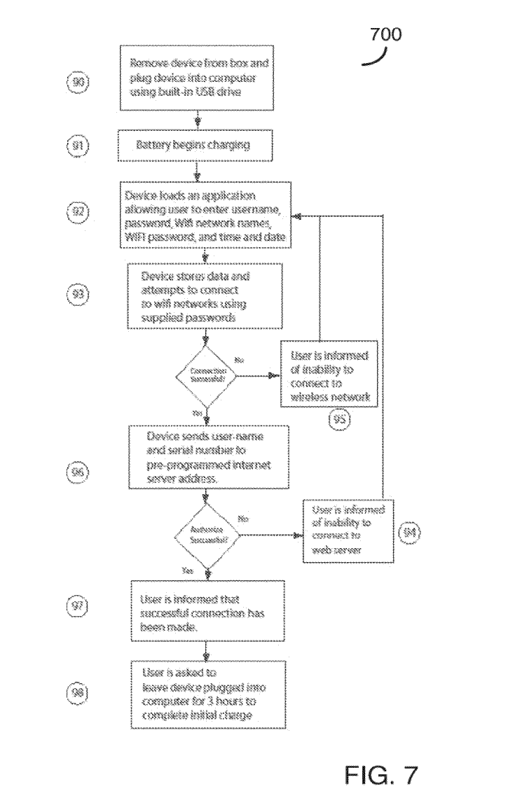

FIG. 7 illustrates one exemplary process that may be carried out for initializing a fertility data collection device and a separate personal computer (or other suitable computing device) to be used with the fertility data collection device. The initialization process 700 of FIG. 7 begins in block 90, in which a user removes the fertility data collection device from packaging and plugs the device into a computer using a built-in USB port. In block 91, the battery of the collection device begins charging and a processor of the collection device displays a charging battery icon on the screen.

The collection device, in this embodiment, stores software executable on the computer. This software may include instructions for configuring the collection device and/or the computer. When plugged in, a programmable processor and memory chip allow the collection device to interact with the computer like a standard USB flash memory disk. More particularly, when plugged into a computer, the thermometer appears as a disk on the computer. With some computers, the software executable on the computer may open and run automatically on the computer when the collection device USB connector has been energized by the computer and the collection device has been recognized by the computer. Alternatively, a user can view, via the computer, files stored on the collection device and manually open this software stored on the collection device. It should be appreciated, however, that embodiments are not limited to storing the configuration software on the collection device. In other embodiments, the configuration software may be provided separately, such as via a compact disc (CD), or the configuration software may be downloaded from the Internet.

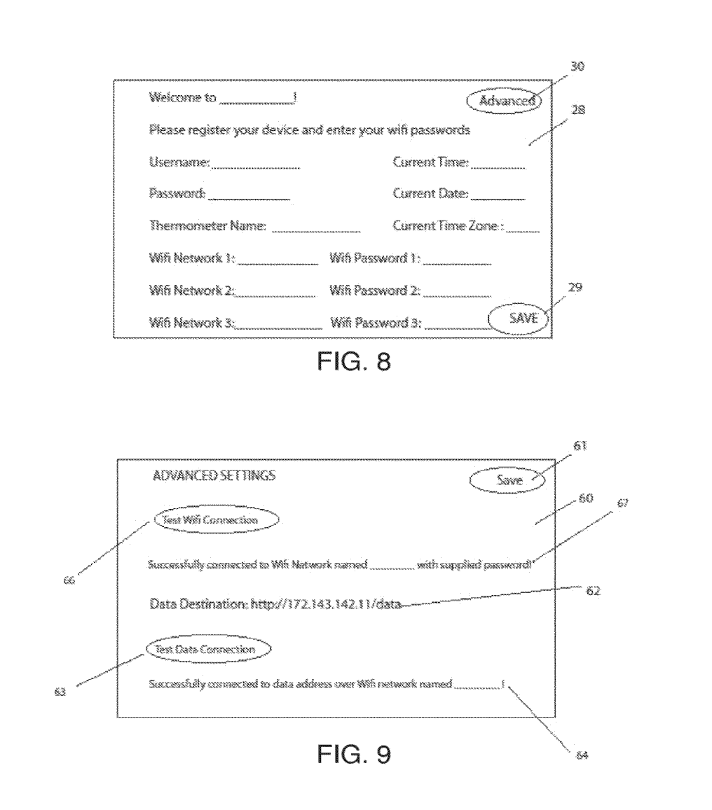

In block 92, regardless of how the configuration software is provided to the computer, the software begins executing on the computer. The software loads a screen that allows a user to initialize and personalize the collection device. FIG. 8 shows one such exemplary screen 28 which may be used in block 92 to input information. The screen 28 allows a user to provide identification information, for example, for the user, for the thermometer, and/or for nearby wireless networks.

As shown in FIG. 8, a field on the screen 28 allows the user to type in a username and password. The username and password may be used to communicate data to and/or from a remote server and/or remote database. As discussed in further detail below, in some cases, the username and password may be sent as part of a transmission of fertility data. The entered username and password must match a username and password combination configured on the remote database for transmission of data to occur. In other embodiments, however (discussed in detail below), a serial number for the collection device is instead transmitted with fertility data and used to identify the device and the user.

Also on the initialization screen 28 is a field that allows naming of the collection device. Such naming may instill a sense of intimacy between the collection device and the user. The name supplied by the user is used to identify the collection device on the computer and, in some cases, on remote servers.

In embodiments in which collection devices communicate fertility data via WLANs, the collection devices may be configured to use multiple WLANs and may be configured to use WLAN securely. Accordingly, in screen 28, multiple fields are provided for the user to enter the identifiers and passwords of multiple WLANS that will be in range of the collection device. For example, these might be a network at the user's home, a network at the user's partner's home, and/or a network at a user's place of employment. Entering multiple WLAN identifiers and passwords allows the collection device to establish an Internet connection at multiple locations, which allows fertility data to be transferred from these locations. Alternatively, the software may allow the device to detect all available wireless networks and have the user pick from a list of the networks she wishes the device to use to communicate.

The screen 28 may also allow the user to configure the collection device with the current time, date and time zone. In alternative embodiments, however, the collection device may synchronize with a time-server.

Other configuration information may be provided in other embodiments. For example, in some embodiments, the initialization screen 28 may also contain an option for the collection device to display and transmit temperature in degrees Celsius or Fahrenheit. As another option, the user may be able to enable or disable sounds produced via a speaker of the collection device, such as sounds indicating successful collection or transmission of fertility data.

An advanced tab 30 may display an additional screen that enables a user to input other settings. An example of such a screen 60 is shown in FIG. 9. Using the screen 60, a user can change the destination to which the collection device will transmit fertility data wirelessly, such as via a home wireless network. The collection device may be pre-programmed with an Internet server address to which fertility data may be sent following the establishment of a successful connection with a wireless network. Such an Internet server may be one associated with a service for graphing the fertility data. However, should the user wish to change the Internet server address to which the data is sent, the user is able to do so via the screen 60, by entering a different address in the field 62. A button 63 also may be provided in the screen 60 to enable the user to test whether the collection device is able to connect to a computer that has the address input into field 62. When the data connection is tested in response to the user operating button 63, a message 64 may be displayed showing the result of the test.

Additionally, a button 66 may be provided to enable the user to test whether the collection device is able to form a connection with one of the networks with which the device is configured, such as a network specified in screen 28 of FIG. 8. During or following the test, information on the status of the test may be displayed in message 67.

For collection devices that communicate over cellular data networks, a message 67 “now testing connection with cellular network” may be displayed while the thermometer attempts to establish itself on the network using its SIM card ID and cellular transmitter/receiver. If no connectivity can be established, a message 67 may be displayed to that effect.

For collection devices that communicate using Bluetooth, a message 67 “now testing connection with Bluetooth network” may be displayed while the thermometer attempts to establish a Bluetooth connection with Bluetooth enabled devices in range (e.g., a WiFi device or smart phone). In some cases, to connect with a WiFi device like an iPad or a smart phone, the user must first download an application onto the WiFi device or smart phone to allow Bluetooth connectivity with the collection device and forwarding of data to the appropriate data destination. If no connectivity can be established, a message 67 may be displayed to that effect. If a connection is established, the user may enter a verification code in the configuration tool to verify the connection between the two devices, as is standard for Bluetooth devices.

For embodiments communicating on WLAN networks (e.g., home wireless networks), a message 67 “Now testing connection with wireless networks” may be displayed while the collection device tests the connection with the specified networks using the specified passwords. Once a connection is established, the thermometer may display a message 67 “Connection has been established with the network (NAME) using the password provided”. If no connectivity can be established, the application may display a message 67 to that effect.

When the user is finished entering data or testing connections in the advanced screen 60, the user may press the save button 61 to return to the screen 28 of FIG. 7. Similarly, the user may end an interaction with the initialization screen 28 of FIG. 7 by operating the “Save” button 30.

Returning to FIG. 7, once the user operates the Save button 30, the process 700 continues to block 93. Upon operating the Save button 30, initialization data provided by the user is written into memory of the collection device. In the embodiment of FIG. 7. the collection device also automatically tests its operations based on the new configuration, such as by attempting to form a connection with all wireless networks in range with which the collection device is configured to communicate.

A determination may then be made as to whether a connection can be successfully established with at least one wireless network. If not, then in block 95 the user may be alerted of an inability to connect to the wireless networks in range and the process 700 returns to block 92 for the user to update the configuration information regarding wireless networks.

If, however, a connection was successfully established, then in block 96 the collection device attempts to communicate with the data destination with which the collection device is configured. The collection device may communicate any suitable information to the data destination, including the username and password of the user and/or a serial number of the collection device. A determination is then made regarding whether the collection device successfully formed a connection (e.g., by being authenticated by an authentication process and/or by receiving some response from the data destination). If not, then in block 94 the user is alerted of the inability to connect to the data destination and the process 700 returns to block 92 for the user to update the configuration information regarding the username and password and data destination.

If, however, a connection was successfully established to the data destination, then in block 97 the user is informed that the connection was successfully established. The user then may be requested in block 98 to leave the collection device connected to the computer for at least three hours to fully charge the battery.

Following presentation of the message in block 98, the initialization process 700 ends. The fertility data collection device is fully configured. Once charging is complete (which may be indicated by a solid battery icon on the screen), the user can detach the collection device from the computer (using, for example, the same process one would use to detach a standard flash memory stick from the computer). Once detached, the collection device may turn off. Following detachment, the thermometer may be ready for use in collecting fertility data and transmitting the fertility data to a remote destination.

When the data destination receives the username and password and serial number transmitted in block 96 of FIG. 7, the data destination may create a new user account for a database corresponding to the username, so that fertility data transmitted later may be stored in association with the user account. The serial number may also be stored with the user account, so that when fertility data is transmitted with a serial number, the serial number may be used to identify the user account to which the fertility data should be matched. In some embodiments, the data destination may also determine whether the username and password matches an existing user account and, if so, associate the serial number with the existing user account rather than create a new user account. In this way, a user can associate multiple fertility data collection devices with a user account, so that the user may keep a collection device at multiple locations (e.g., at home, at work, and at a partner's home).

A fertility data collection device may be used in many different ways to collect fertility data and transmit the fertility data to a data destination. Two general examples will be discussed first, followed by a process that may be implemented by some exemplary collection devices for interacting with a user.

In some cases, users will measure cervical fluid for a female and enter the measurement into the collection device at night for the current day, or in the morning for the previous day. In these cases, a typical use of the collection device could be one of the following. In these examples, the user interface of the exemplary embodiment of FIGS. 4A and 4B is discussed. Embodiments are not, however, limited to using the exemplary interface of FIGS. 4A and 4B.

In the Evening:

A user picks up the collection device from the bedside table before she goes to bed and presses an on/off button (e.g., button 424 of FIG. 4A). By default, when the thermometer turns on, the selected date is the present day and the screen backlight turns on. The user enters her cervical fluid reading for that day (which she may have obtained by passing the cervical fluid between her fingers and selecting a consistency and amount that she felt closely matched the observed cervical fluid) by pressing a cervical fluid button corresponding to a cervical fluid consistency an appropriate number of times or by rotating a cervical fluid button wheel to represent the desired consistency and pressing the wheel 1, 2 or 3 times to indicate the desired amount. For example, if a user observes a large amount of “Creamy” cervical fluid, she presses the “Creamy” button three times. Similarly, a small amount of “Sticky” cervical fluid can be indicated by pressing the “Sticky” button once. In the display screen, when the cervical fluid buttons are pressed, one, two, or three dots may appear next to the actuated button to indicate the amount of the type of fluid. The user then presses the send button (e.g., button 412 of FIG. 4A) and the thermometer sends its information over the pre-programmed home wireless network. The status indicator on the screen provides feedback to the user that the transmission was made and was received. After 30 seconds of no input, the collection device may turn off. When she is done, the user may place the collection device screen facing downwardly on her bedside table or she may press the on/off button to turn off the device.

In the Morning:

A user picks up the collection device from the bedside table after she awakens and presses the on/off button. The collection device powers on, the screen backlight turns on, and when the collection device is ready to accept a temperature measurement, the screen provides a display such as “______”. The user takes her temperature. The collection device produces a “beep” from a speaker when the measurement has been recorded. If the user neglected to enter a cervical fluid measurement from the night before, the user may do so now. The device is set up to record a measurement taken at the current time. To enter a cervical fluid measurement for the previous day, the user may press a “previous day” button (e.g., button 406 of FIG. 4A), which displays the date of the previous day. Upon operation of the button, the user may then enter a cervical fluid measurement as above and press the send button to transmit collected fertility data wirelessly. The collection device transmits all fertility data collected since the last transmission, including temperatures, time and date of temperature readings, cervical fluid measurements, date of cervical fluid measurements, intercourse, and date of intercourse to the data destination. The collection device may turn off after 30 seconds of no input. Once the user is done with the collection device, the user may place the collection device on a table or the like, screen down. By placing the device screen-down, a solar cell on a side opposite the screen is exposed to sunlight and charges the battery using the ambient light in the room during the day.

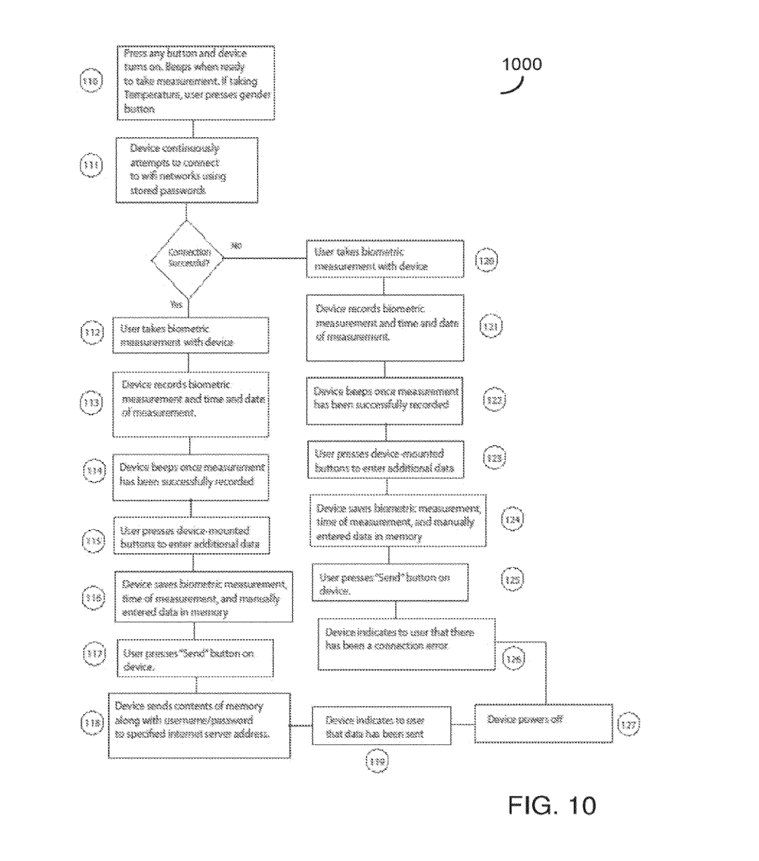

FIG. 10 illustrates in detail one exemplary process 1000 for collecting and transmitting fertility data using a fertility data collection device.

The process 1000 begins in block 110, in which the user presses the on button to turn on the device. The device may carry out any suitable power-on operations and produce a “beep” from a speaker when ready to collect fertility data.

In block 111, the device begins to attempt to connect to wireless networks within range and with which the collection device is configured to communicate. If the collection device is not able to connect, in some embodiments the collection device may not immediately produce an error indicating that the connection is not successful. Instead, the collection device may continue collecting and storing fertility data until the user operates the “send” button to transmit fertility data.

Whether the device successfully established a connection, or is still trying to create a connection, the user takes temperature reading in one of blocks 112 or 120. The temperature may be taken in any suitable manner, including orally, anally, vaginally, or under an armpit, as long as the user consistently takes the temperature at the same location over time. To take a temperature orally, the thermometer of the collection device may be inserted into the mouth and gripped by the teeth. The collection device may output a “beep” via a speaker when the collection device begins and/or ends the temperature measurement. In some cases, the collection device may begin the temperature measurement when the thermometer senses a temperature above 90 degrees and may stop when the temperature sensed by the thermometer settles to a constant temperature for a predetermined period. The temperature sensed then may be displayed on the screen along with the time the temperature was sensed. In blocks 113, 121, the collection device records the temperature measurement alongside the time and date of the measurement and, in blocks 114, 122, beeps once the measurement has been successfully recorded.

The user may also enter cervical fluid observations by pressing additional buttons of the collection device in blocks 115, 123. As discussed above, cervical fluid types range in consistency from “Menstruation” to “None” to “Sticky” to “Creamy” to “Egg-white” in order of increasing fertility. In some embodiments, a user may change these labels to ones that more closely suit the user, but the fertility scale may remain the same. To measure consistency and amounts of cervical fluid, the user places the cervical fluid between the user's fingers and selects which category and amount most closely corresponds to the observed cervical fluid. The user may then operate the button of the collection device corresponding to the selected consistency, the number of times that corresponds to the observed amount. When the data is input to the collection device in blocks 116, 124, the device saves all data and the time the input was received.

In blocks 117, 125, the user presses the “Send” button to convey the collected fertility data to a remote destination, such as a web server or Internet-connected data store. If the collection device was successful earlier, or has since been successful, in connecting to the data destination, the collection device sends the collected fertility data (including new fertility data as well as any previously-collected and not yet transmitted fertility data) along with the serial number for the collection device. Details of one possible way that this transfer is carried out are provided below. Once the transfer is complete, the collection device indicates as such in block 119 and powers off in block 127.

If, however, the device has not been successful in establishing a wireless connection, a connection error indication may be shown to the user in block 126 and the device powers off in block 127. If the user wishes to know more about the error, the user can plug the device into a computer and use the device application to troubleshoot the connection problem.

The transfer of fertility data between a fertility data collection device and a data destination (e.g., a web server or database) may be carried out in any suitable manner. In some embodiments, when she wishes to send stored fertility data to a data destination, the user presses a “Send” button on the collection device that tells the collection device to send the contents of the memory (i.e., one or more units of fertility data) to the data destination programmed in the configuration application or pre-programmed on the collection device. When the “Send” button is pressed for 1 second, the thermometer attempts to connect to a wireless network if a connection has not already been established. If a successful connection is made, the following series of communications may take place between the collection device and a Data Destination Server (DDS):

i. Thermometer sends serial number and/or username and password to DDS

ii. DDS checks serial number against numbers stored in a database. If the serial number is in the database and associated with a user account, an “Authentication OK” message is sent to the collection device. In the case that username and password are also sent, the serial number may be used to retrieve a username and password associated with the collection device to determine if there is a match with the username and password provided by the user. If there is a match, the DDS sends an “Authentication OK” message to the collection device. If the serial number is not in the database or there is not a match between the usernames and passwords, the DDS sends an “Authentication Error” message to the collection device.

iii. If the collection device receives an “Authentication Error” message from the DDS, the collection display lights a red LED and displays “Authentication Error” on the screen for 5 seconds before turning off.

iv. If, however, the collection device receives an “Authentication OK” message from the DDS, the collection device establishes a secure connection with the DDS and sends one or more units of fertility data to the DDS, including each reading and the time of each reading.

v. Each measurement transmitted by the collection device is sent in a packet containing a type of the measurement (temperature, cervical fluid consistency, cervical fluid amount, etc.) and the time and date of the measurement (Minutes, Hours, Day, Month, Year) and the value of the measurement itself.

vi. Once all measurements have been sent by the collection device, the DDS repeats back all measurements to the thermometer.

vii. If the collection device receives back a non-identical measurement from the DDS, the collection device tries to transmit the measurement again

viii. In some embodiments, as the information is being transmitted the collection device blinks an amber-colored LED or displays a progress icon on the screen.

ix. Once all measurements have been transmitted, the collection device terminates the session with the DDS, and terminates the wireless connection.

x. If information cannot be transmitted for any reason after 3 minutes, the collection device displays an error message on the screen.

In some embodiments, the user need not transmit fertility data immediately upon collecting the fertility data. Rather, if the user makes measurements but does not press the send button, the collection device records the measurement and turns off after 30 seconds of inactivity or when the off button is pressed. This allows the user to take and record measurements when out of range of a wireless network.

A data destination may take any suitable action upon receiving fertility data from a fertility data collection device. In some embodiments, the data destination may store received fertility data in a user account and then chart the fertility data in a single graph. The single graph, upon request, may be inserted into a web page and transmitted to the user from whom the fertility data was received. In some embodiments, by default the fertility data may only be viewable by the user from whom the data was received. The user may elect, however, to share her fertility data with others. For example, fertility data may be shared between spouses or partners, or shared with a medical professional. When data is shared in this way, the other person with whom the data is shared may be able to retrieve the graph in the web page upon request. Sharing of fertility data stored in the database may be carried out in any suitable manner, including using known techniques.

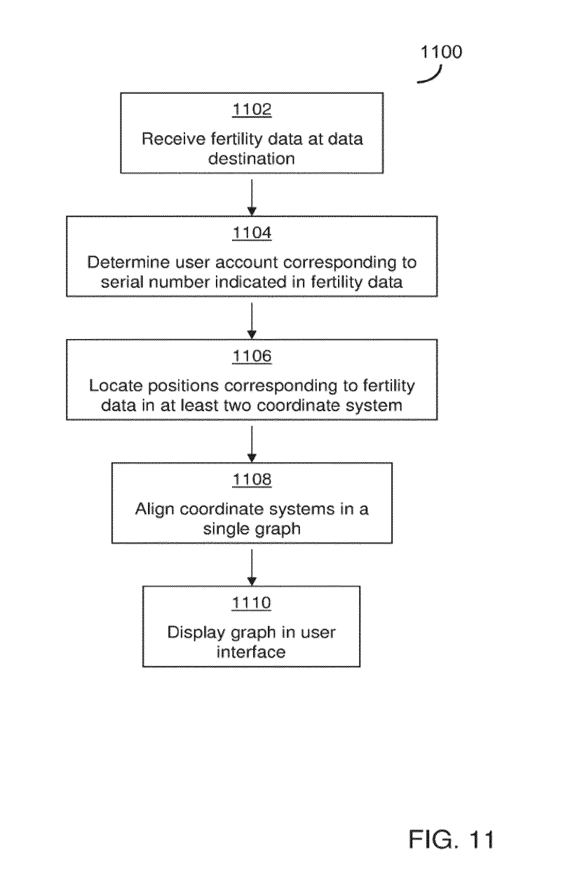

The data destination server may carry out any suitable process for translating fertility data into a graph, such as the graph 100 of FIG. 1. FIG. 11 shows one exemplary process that may be used in some embodiments to create a graph from fertility data received from a collection device. Embodiments are not limited to implementing a process for creating a graph or, in embodiments that implement such a process, to implementing any particular process.

The exemplary process 1100 of FIG. 11 begins in block 1102, in which fertility data is received by a data destination from a collection device. The data may have been relayed to the data destination via one or more wireless networks and the Internet. The received fertility data may include information on one or more fertility characteristics, including temperature, consistency of cervical fluid, and amount of cervical fluid. In addition, the fertility data may include a serial number of the collection device and a time the fertility data was collected. Upon receiving the fertility data, in block 1104 the data destination may use the serial number to identify a user account with which the collection device is associated and may store the received fertility data in association with the user account.

In addition, in block 1106, the fertility data (including the fertility data received in block 1102 alone or together with previously-received fertility data) may be analyzed to develop a chart. In block 1106, each of the units of fertility data may be analyzed and a position corresponding to the fertility data in one, two, or more two-dimensional coordinate systems is identified. The fertility data typically include information on characteristics to which the coordinate systems relate. Accordingly, in block 1106, coordinates corresponding to the fertility data are located.

In block 1108, the two or more coordinate systems are superimposed one over the other in a single graph, such as FIG. 1. For example, coordinates that indicate high fertility (like reference 9 of FIG. 1) are aligned. By doing so, lines indicating variations in characteristics in each of the coordinate systems will be superimposed at locations corresponding to the level of fertility, allowing a user to more clearly identify times of high and low fertility.

In block 1110, once the coordinate systems are superimposed in the graph, the graph may be displayed in a user interface. The graph may be displayed with the fertility data, such as dots at the positions on the graph corresponding to the received fertility data and/or lines connecting those positions. In addition, any other suitable fertility data may be displayed in the graph. For example, in embodiments where data regarding intercourse is collected, some indication may be provided regarding days on which intercourse occurred, such as an icon along the time axis. In some embodiments, the icon may be a heart. A solid heart indicates when the intercourse was unprotected and an outline of a heart indicates when the intercourse was protected. Other fertility data may be displayed in the graph in any suitable manner. The graph of block 1110 may be displayed on any suitable user interface. In some cases, for example, the graph may be inserted into a web page and displayed to a user via a web browser.

Once the graph is displayed, the process 1100 ends.

In each of the examples above, a data destination receives fertility data via the Internet from a fertility data collection device. However, the invention is not so limited. In some embodiments, a data destination may additionally or alternatively receive fertility data via a user interface of the data destination. For example, in some embodiments, a user may be able to input fertility data via the same web page in which the single graph of the fertility data is displayed. For example, by interacting with the graph, the user may be able to add fertility data to the graph.

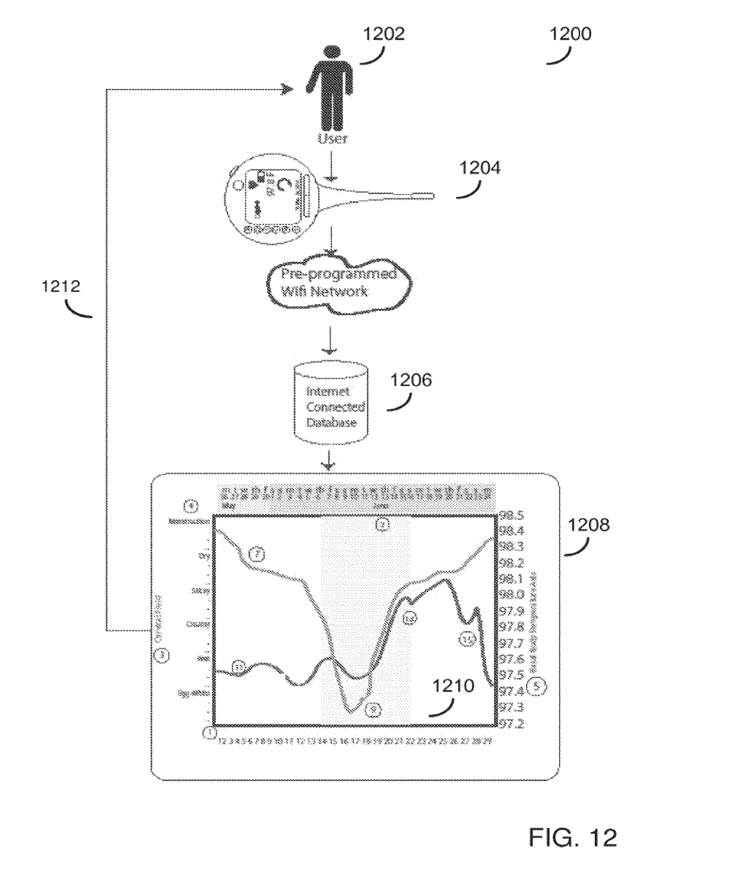

Discussed above are various components of exemplary systems for collecting and tracking information regarding fertility of a female. In some exemplary embodiments, the system includes four main components, which are illustrated in FIG. 12. In the system 1200, a user 1202 interacts with a wireless fertility data collection device 1204 to take temperature measurements and enter cervical fluid observations, and occurrences of intercourse. The collection device 1204 communicates with an Internet-connected database 1206 over a user-programmed wireless network, like a home wireless network. One or more user applications 1208 can also run on top of the database and provide some meaning to data communicated to the database 1206. The user applications can execute in any suitable redundant manner, including by being delivered by a web-server through an Internet browser or through a custom application on a smart-phone, iPad, or other device that can connect to the Internet and that has a screen. In some cases, within each user application, fertility data, like temperatures and cervical fluid measurements, may be displayed on the same graph 1210 with more fertile cervical fluid represented at the bottom of the left Y-axis. The graph may be displayed via a web browser 1212 to the user 1202.

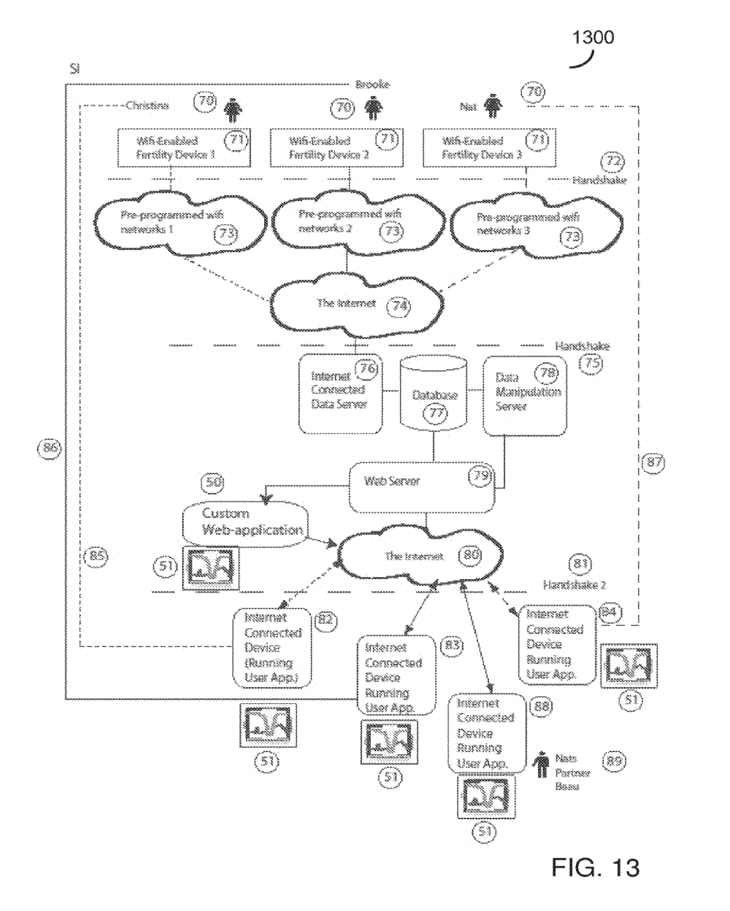

The representation of the system 1200 of FIG. 12 is simple, with the components illustrated at a high level. However, the simple system 1200 can be implemented and used in complex ways while making the collection and tracking of fertility information simpler for users. FIG. 13 shows an example of such a complex system and is discussed below in the context of four users, three with a fertility data collection device in communication with a data destination and one with whom fertility data is shared.

In the system 1300 of FIG. 13, each of the three women 70, Christina, Brooke, and Nat, has a wireless fertility data collection device 71 that each uses to take and record her fertility characteristics, including temperature and cervical fluid observations, as well as the times of all measurements. Each user has programmed her device to communicate over a WiFi network 73 (e.g., a home wireless network) that she has chosen to use with her device 71. As the users enter data into their devices 71, a first authentication “handshake” 72 takes place when each device attempts to connect to these wireless networks 73. The handshake is successful when the device sends the correct password to make a connection with the configured WiFi network, and a successful handshake enables the devices to communicate using the networks.

Following a successful handshake, the devices communicate across these WiFi networks to reach the Internet 74. Here a second authentication “handshake” 75 takes place between the device and an Internet-connected data server 76. During the handshake, each device 71 sends a username and password and/or serial number to the data server 76. In response to receiving these credentials, the data server checks the credentials against credentials stored in the database 77. If the credentials match, the second handshake is successful and the devices 71, are allowed to communicate with the database 77. When allowed, each device 71 sends fertility data through the data server 76 to be stored in the database 77.

The database 77 is connected to both a web server 79 and a data manipulation server 78. The manipulation server 78 may make certain calculations to determine whether any received data is questionable or suspect, or even wrong. For example, a temperature measurement may be too high or too low based on past data collected for the user. Data identified as suspect by the manipulation server 78 may still be processed and displayed in a chart, but could be flagged in the interface as suspicious. In addition, a web server 79 is connected to the database 77 and data manipulation server 78 and responds to requests to serve data. The web server 79 is connected to the Internet 80. A user-interface web application 50 runs on the web server 79, allowing users to access a user interface with any Internet-connected device running an Internet browser. For example, Christina uses her computer 82 to access her data using her browser. Users can also access their data using a custom user-interface application running on their internet-connected device. For example, Brooke uses her iPhone 83 running a custom iPhone application to access her data through the web server 79, and Nat uses her iPad 84 running a custom iPad application to access her data through the web server 79.

Whatever device is used, the web application 50 and device-specific user applications use the same display method. The entire process creates a feedback loop for each user from the measurement of her data, through transmission, storage, manipulation and finally presentation of her data. Christina's feedback loop 85, Brooke's feedback loop 86, and Nat's feedback loop 87 allow each one to easily and quickly make decisions about fertility.

Additionally, users can share their data, or connect more than one wireless fertility device to their accounts. For example, if Nat decides to share her data with her partner Beau 89, she sends him an invitation with a login and password that gives him access to her data. Beau can use any Internet-connected device 88 to access Nat's data.

In each of the examples described above, fertility data collected by a fertility data collection device or collected in any other manner can be displayed to a user such that the user is able to determine a fertility of the female to which the fertility data relates. In some cases, however, not all fertility data collected may be used. For example, a user may be able to determine whether to transmit fertility data collected. This would allow a user to collect some fertility data and determine, prior to sending, if the fertility data is in error. For example, if a user finds that the female's temperature varies from an expected temperature, then the user may elect not to use the temperature in determining a fertility of the female. This may also allow the user to collect data in other contexts. For example, if the user would like to collect a temperature of another person other than the female (e.g., another family member), the user could use the same fertility data collection device and then not transmit the collected data.

Accordingly, a fertility data collection device may be implemented that does not transmit fertility data from the device until a user instructs the data to be transmitted. As another example, a user may be asked, following collection of fertility data, to mark data to be sent or not sent. In one exemplary fertility collection device, upon collection of fertility data the user may be asked to input whether that data should be transmitted with a next transmission from the device. In the context of the exemplary device 400 of FIGS. 4A and 4B, upon collection of a temperature, the user may be prompted to push the send button 412 within some period of time following collection of the temperature (e.g., 30 seconds). If the send button 412 is not pushed within that period, then the next time fertility data is transmitted from the device, that temperature may not be transmitted. The display 414 of the device may be adapted with a display area that indicates fertility data that has or has not been transmitted.

Techniques operating according to the principles described herein may be implemented in any suitable manner. Included in the discussion above are a series of flow charts showing the steps and acts of various processes that can be used for collecting, tracking, and displaying fertility data. The processing and decision blocks of the flow charts above represent steps and acts that may be included in algorithms that carry out these various processes. Algorithms derived from these processes may be implemented as software integrated with and directing the operation of one or more multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that the flow charts included herein do not depict the syntax or operation of any particular circuit, or of any particular programming language or type of programming language. Rather, the flow charts illustrate the functional information one of ordinary skill in the art may use to fabricate circuits or to implement computer software algorithms to perform the processing of a particular apparatus carrying out the types of techniques described herein. It should also be appreciated that, unless otherwise indicated herein, the particular sequence of steps and acts described in each flow chart are merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, or any other suitable type of software. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations needed to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package or web application. These functional facilities may, in alternative embodiments, be adapted to interact with other, unrelated functional facilities and/or processes, to implement a software program application or web application.

Some exemplary functional facilities have been described herein for carrying out one or more tasks. It should be appreciated, though, that the functional facilities and division of tasks described are merely illustrative of the type of functional facilities that may implement the exemplary techniques described herein, and that embodiments are not limited to being implemented in any specific number, division, or type of functional facilities. In some implementations, all functionality may be implemented in a single functional facility. It should also be appreciated that, in some implementations, some of the functional facilities described herein may be implemented together with or separately from others (i.e., as a single unit or separate units), or some of these functional facilities may not be implemented.

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CI)) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner, including as computer-readable storage media of FIG. 3A (i.e., as a portion of a computing device 300) or as a stand-alone, separate storage medium. As used herein, the term “computer-readable media” (also called “computer-readable storage media”) refers to tangible storage media. Tangible storage media are non-transitory and have at least one physical, structural component. In a “computer-readable medium.” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system, including the exemplary computer system of FIG. 9 or 10. Functional facilities that comprise these computer-executable instructions may be integrated with and direct the operation of a single multi-purpose programmable digital computer apparatus, a coordinated system of two or more multi-purpose computer apparatuses sharing processing power and jointly carrying out the techniques described herein, a single computer apparatus or coordinated system of computer apparatuses (co-located or geographically distributed) dedicated to executing the techniques described herein, one or more Field-Programmable Gate Arrays (FPGAs) for carrying out the techniques described herein, or any other suitable system.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and are therefore not limited in their application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

Having thus described several aspects of at least one embodiment, it is to be appreciated that various alterations, modifications, and improvements will readily occur to those skilled in the art. Such alterations, modifications, and improvements are intended to be part of this disclosure, and are intended to be within the spirit and scope of the principles described herein. Accordingly, the foregoing description and drawings are by way of example only.

1. A method for charting fertility data regarding a female, the method comprising:
receiving the fertility data regarding the female, the fertility data comprising a temperature of the female and at least a consistency of a cervical fluid of the female;
superimposing at least two two-dimensional coordinate systems in a single graph of a graphical user interface, the at least two two-dimensional coordinate systems comprising a first coordinate system having a temperature dimension and a time dimension and a second coordinate system having a cervical fluid dimension and a time dimension, the superimposing comprising identifying first coordinates corresponding to the temperature in the first coordinate system and identifying second coordinates corresponding to the cervical fluid consistency in the second coordinate system, wherein superimposing the at least two two-dimensional coordinate systems in the single graph comprises aligning the at least two two-dimensional coordinate systems such that coordinates indicating high fertility of the female in each of the at least two two-dimensional coordinate systems are closely aligned; and
displaying the fertility data in the graphical user interface.

2. The method of claim 1, wherein receiving the fertility data further comprises receiving an input relating to an amount of the cervical fluid of the female.

3. The method of claim 2, wherein identifying second coordinates corresponding to the cervical fluid consistency comprises identifying the second coordinates based at least in part on the amount of the cervical fluid.

4. The method of claim 3, wherein identifying second coordinates in the second coordinate system comprises identifying a coordinate in the cervical fluid dimension based on both the consistency and the amount of the cervical fluid, wherein the cervical fluid dimension includes a plurality of sections corresponding to types of consistencies of cervical fluid and includes, within each section, values corresponding to amounts of a corresponding consistency of cervical fluid.

5. The method of claim 4, wherein the cervical fluid dimension is arranged such that the plurality of sections and the values included within each section are arranged in an order corresponding to a level of fertility indicated by the consistencies and amounts of cervical fluid.

6. The method of claim 4, wherein superimposing the at least two two-dimensional coordinate systems in the chart comprises orienting the second coordinate system in the chart such that there is a first distance in the chart between a first coordinate corresponding to a highest amount of cervical fluid of a first consistency and a′second coordinate corresponding to a lowest amount of cervical fluid of a second consistency is greater than a second distance in the chart between two adjacent coordinates corresponding to two different amounts of a same consistency of cervical fluid.

7. The method of claim 1, wherein displaying the fertility data in the graphical user interface further comprises:
retrieving from a data store previously-received fertility data for the female;
indicating in the graph of the graphical user interface at least one coordinate corresponding to the previously-received fertility data in each of the at least two two-dimensional coordinate systems;
drawing a line in the graph linking a first coordinate and a second coordinate indicated in the graph for the first coordinate system of the at least two two-dimensional coordinate systems, the first coordinate corresponding to data of the previously-received fertility data and the second coordinate corresponding to data of the fertility data; and
drawing a sequence of bars in the graph extending between an axis of the single graph and coordinates of the second coordinate system of the at least two two-dimensional coordinate systems, the sequence of bars extending between the axis and the coordinates comprising a first bar extending between the axis and a third coordinate of the second coordinate system and a second bar extending between the axis and a fourth coordinate of the second coordinate system, the third coordinate corresponding to data of the previously-received fertility data and the fourth coordinate corresponding to data of the fertility data.

8. The method of claim 7, wherein drawing the line and drawing the sequence of bars in the graph comprises drawing the line and the sequence of bars such that, when the fertility data indicates a high fertility of the female, the line and heights of bars of the sequence of bars converge in the graph.

9.-11. (canceled)

12. The method of claim 1, wherein receiving the fertility data further comprises receiving a particular time the fertility data was collected from the female.

13.-17. (canceled)

18. The method of claim 1, wherein receiving the fertility data comprises receiving at least one communication from a device comprising a thermometer and at least one user interface for inputting at least a consistency and amount of cervical fluid.

19.-35. (canceled)

36. A system for managing fertility data regarding a female, the system comprising:
at least one device to accept as input fertility data regarding the female, each of the at least one device comprising
a thermometer to measure the basal body temperature of the female, and
a user interface to accept input regarding a consistency and an amount of cervical fluid of the female, the user interface comprising a plurality of interaction elements each corresponding to a type of consistency of cervical fluid; and
at least one server to store fertility data received from the at least one device and to generate a graph for a fertility cycle of the female, the graph comprising a sequence of bars and a line corresponding to fertility data received from the at least one device, the sequence of bars indicating variations in consistency and amount of cervical fluid of the female over time and the line indicating variations in temperature of the female over time,
wherein the at least one device and the at least one server are connected via at least one communication network, the at least one communication network comprising a wireless network and the Internet.

37. The system of claim 36, wherein the at least one server is configured to store fertility data comprising information regarding a vaginal sensation of the female and is configured to generate the graph comprising an indication of the vaginal sensation over time.

38.-39. (canceled)

40. The system of claim 36, wherein the at least one server is configured to generate a second graph for a second fertility cycle of the female.

41. The system of claim 36, wherein the at least one server is configured to generate a second graph for a fertility cycle of a second female.

42. The system of claim 36, wherein the at least one server is adapted to generate the graph as a part of a web page.

43. The system of claim 42, further comprising a client computer,
wherein the at least one server is adapted to transmit the web page comprising the graph to the client computer.

44. The system of claim 36, wherein the at least one server is adapted to generate the graph comprising the at least two lines such that points in the at least two lines corresponding to high fertility of the female are closely aligned.

45. The system of claim 36, wherein the at least one server comprises fertility data for a plurality of females associated with a plurality of user accounts, and wherein the at least one server stores the fertility data for the plurality of females with access restrictions such that a graph illustrating fertility data for each female is only viewable by specified user accounts, and
wherein the at least one server is adapted to change access restrictions associated with fertility data for a particular female in response to a request from a user to change the access restrictions.

46.-49. (canceled)

50. A method of distributing fertility data regarding a female, the method comprising:
receiving the fertility data regarding the female, the fertility data comprising at least one temperature of the female and at least one indication regarding a cervical fluid of the female;
storing the fertility data in a fertility database in association with a user account associated with the female;
restricting access to the fertility data on the fertility database to the user account;
receiving an instruction from a user for the user account to share the fertility data with at least one second user account; and
permitting access to the fertility data on the fertility database by the at least one second user account.

51. The method of claim 50, wherein the fertility data comprises information regarding at least one fertility characteristic from a group of fertility characteristics consisting of basal body temperature, cervical fluid consistency, and cervical fluid amount.

52. (canceled)
